(19) United States
(12) Patent Application Publication
(10) Pub. No.: US 2004/0162247 A1
(43) Pub. Date: Aug. 19, 2004

(54) Therapeutic agent or osteoporosis comprising an active ingredient of quercetin derivatives

(76) Inventors: Chung-Sook Kim, Seoul (KR); Hye-Kyung Ha, Seoul (KR); Kye-Yong Song, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/783,084

(22) Filed: Feb. 19, 2004

Related U.S. Application Data
(63) Continuation of application No. 10/070,047, filed on Feb. 22, 2002, now abandoned.
Application No. 10/070,047 is a 371 of international application No. PCT/KR01/00368, filed on Mar. 9, 2001.

(30) Foreign Application Priority Data
Aug. 14, 2000 (KR) 10-2000-0046916

(51) Int. Cl. A61K 31/7048; A61K 31/353

(52) U.S. Cl. 514/27; 514/456

(57) ABSTRACT

The present invention relates to a therapeutic agent for osteoporosis which comprises an active ingredient of quercetin derivatives. The quercetin derivatives of the invention can be practically applied for the treatment and prevention of osteoporosis, since they effectively inhibit osteoclast proliferation and stimulate osteoblast proliferation more than conventional therapeutic agents for osteoporosis, and increase trabecular bone area highly without changing hormone level in body and untoward effects on hematopoietic function and immune system.

### BACKGROUND OF THE INVENTION

1. Field of the Invention

The present invention relates to a therapeutic agent for osteoporosis which comprises an active ingredient of quercetin derivatives, more specifically, to a therapeutic agent for osteoporosis comprising an active ingredient of quercetin derivatives represented by the following general formula (I) which effectively stimulate osteoblast proliferation and inhibit osteoclast proliferation.

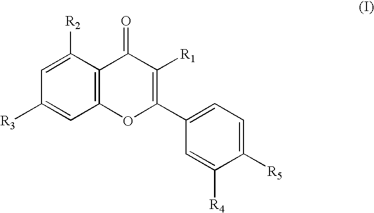

2. Description of the Prior Art

Osteoporosis is a disease characterized by the decrease of bone mass caused by mineral loss and the subsequent expansion of marrow cavity. Bones become brittle with the progress of the disease, and may be easily fractured by a weak impact. Bone mass is affected by various factors such as genetic factors, nutritive condition, changes of hormone level, exercise and life style, and osteoporosis is known to be caused by aging, lack of exercise, low body weight, smoking, low calcium diet, menopause, and ovariectomy. In women, decrease of bone mass begins at the age of 30, and around menopause, concentration of estrogen rapidly decreases and vast amount of B-lymphocytes are accumulated by the similar mechanism to that of B-lymphocyte accumulation by IL-7(interleukin-7), and subsequent pre-B cell accumulation results in increased level of IL-6 which activates osteoclasts, thus, bone mass becomes decreased. In aged people, especially in women of postmenopause, osteoporosis is not the avoidable disease although the severity of the symptom may vary, therefore, many research groups and pharmaceutical companies have made a great deal of efforts for development of therapeutic agents for bone diseases to prevent and treat osteoporosis upon an increase of elderly population.

Therapeutic agents for osteoporosis now being used include estrogen preparations, androgenic anabolic steroid preparations, calcium supplements, phosphate preparations, fluoride preparations, ipriflavone, vitamin D3, etc. In recent years, novel drugs for osteoporosis have been developed, which include Aminobisphosphonate by Merck Co. (U.S.A.) in 1995 and Raloxifene which plays a role of selective estrogen receptor modulator (SERM) by Eli Lilly Co. (U.S.A.) in 1997.

Therapeutic agents for osteoporosis mentioned above are mostly estrogen substances which are known to cause adverse side effects such as cancer, cholelithiasis, and thrombosis. Since long term administration of drug is inevitable in the treatment of osteoporosis, there is a continuing need to develop novel effective agents which can replace estrogen with high safety even when administered for a prolonged period of time.

As estrogen substitutes, phytoestrogens such as soybean isoflavone have been reported. Phytoestrogen, first reported in 1946, was found interim of verifying the cause of clover disease which was named for the high increase (over 30%) of infertility of the sheep fed with red clover (Trifolium subterraneum var. Dwalganup). The cause of clover disease turned out to be an estrogen-like isoflavonoid contained in the plant, hence, the compound obtained from the plant has been named ‘phytoestrogen’. After that, compounds reported as phytoestrogen includes isoflavone compounds such as daidzein, genistein, formononetin, and biochanin A, coumestan compounds such as coumestrol, lignan compounds such as enterolactone, and phenol compounds such as enterodiol. Such phytoestrogens exist mostly in the form of aglycone, 6′-O-acetylglucoside or 6′-O-malonylglucoside, and daidzein and genistein exist in the form of 7-O-glucoside. Among aforementioned compounds, glucosides are known to be hydrolysed with enterobacteria or gastric acid and absorbed in the form of aglycone which is a free isoflavone. The researches have revealed that the said phytoestrogens function similarly to the animal estrogens. That is, the phytoestrogen inhibit proliferation of breast cancer cells by binding to the estrogen receptor and have been reported to be used as the estrogen substitute for the treatment of cardiovascular diseases and other symptoms occurring in the postmenopause women. However, the said phytoestrogens are not widely used for the treatment and prevention of osteoporosis due to the insufficient pharmaceutical effectiveness and high cost required for the isolation and purification from natural products.

Under the circumstances, are strong reasons for developing and exploring alternative compounds with safety and effectiveness for the treatment and prevention of osteoporosis, which can be prepared in an economical manner.

### SUMMARY OF THE INVENTION

The present inventors have made an effort to develop an effective substitute agent for the treatment and prevention of osteoporosis, which is safe and economical, and have found that chemically synthesized quercetin derivatives have activities of stimulating osteoblast proliferation and inhibiting osteoclast proliferation, without any adverse side effects on internal organs, thus, quercetin derivative can be employed as an active ingredient of a therapeutic agent for osteoporosis.

A primary object of the present invention is, therefore, to provide a therapeutic agent for osteoporosis which comprises an active ingredient of quercetin derivatives.

### DETAILED DESCRIPTION OF THE INVENTION

The present invention provides a therapeutic agent for osteoporosis which comprises an active ingredient of quercetin derivatives represented by the following general formula (I) and pharmaceutically acceptable carriers:

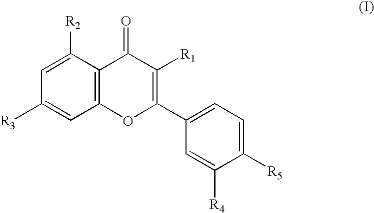

wherein,

R₁ is gentiotriose, glucopyranose, O-arabinofuranose, O-diglucopyranose, O-galactopyranose, O-galactoside-gallate, O-gentiobiose, O-glucopyranose, O-glucuronide, O-neohesperidose, O-rhamnopyranose, O-rutinose, O-sophorose, O-xylopyranose, OCH₃, OH, rhamnogentiobiose, rhamnoglucose or sulfate;

R₂ is OH or O-glucopyranose;

R₃ is OCH₃, OH, O-glucopyranose, O-glucuronopyranose or glucopyranose;

R₄ is OCH₃ or OH; and,

R₅ is OCH₃, OH, O-glucopyranose or O-glucose.

Among the quercetin derivatives represented by general formula (I), well-known compounds are classified as follows: (i) a derivative group of the formula I wherein R₂ to R₅ are OH and R₁ varies, includes quercetin where R₁ is OH, avicularoside where R₁ is O-α-L-arabinofuranose, guiajaverin where R₁ is O-arabinopyranose, hyperoside where R₁ is O-β-D-galactopyranose, isohyperoside where R₁ is O-β-D-galactopyranose, isoquercitrin where R₁ is O-glucopyranose, multinoside A where R₁ is O-[β-D-glucopyranosyl-(1-4)-α-L-rhamnopyranose], multinoside A acetate where R₁ is (6-O-acetyl)-β-D-glucopyranosyl-(1-4)-α-L-rhamnopyranose, quercitrin where R₁ is O-α-L-rhamnopyranose, rutin where R₁ is O-β-D-rutinose, quercetin-3-O-(2″-O-β-D-glucopyranosyl)-α-L-rhamnopyranoside where R₁ is O-(2″-O-β-D-glucopyranosyl)-α-L-rhamnopyranose, quercetin-3-O-(6″-O-galloyl)-glucopyranoside where R₁ is O-(6″-O-galloyl)-glucopyranose, quercetin-3-O-(6′″-O-p-coumaroyl-β-D-glucopyranosyl-(1-2)-α-L-rhamnopyranoside) where R₁ is O-(6′″-O-p-coumaroyl-β-D-glucopyranosyl-(1-2)-α-L-rhamnopyranose, quercetin-3-O-D-glucopyranosyl-(1-6)-β-D-glucopyranosyl-(1-4)-α-L-rhamnopyranoside where R₁ is O-D-glucopyranosyl-(1-6)-β-D-glucopyranosyl-(1-4)-α-L-rhamnopyranose, quercetin-3-O-[2″-O-6′″-O-p-(7′″-O-β-D-glucopyranosyl)coumaroyl-β-D-glucopyranosyl]-α-L-rhamnopyranoside where R₁ is O-[2″-O-6′″-O-p-(7″″-O-β-D-glucopyranosyl)coumaroyl-β-D-glucopyranosyl]-α-L-rhamnopyranose, quercetin-3-O-[6′″-p-coumaroyl-β-D-glucopyranosyl-β-(1-4)-rhamnopyranoside] where R₁ is O-(6′″-p-coumaroyl-β-D-glucopyranosyl-β-(1-4)-rhamnopyranose], quercetin-3-O-[α-L-rhamnopyranosyl (1-2)-α-L-rhamnopyranosyl-(1-6)-β-D-glucopyranoside] where R₁ is O-[α-L-rhamnopyranosyl (1-2)-α-L-rhamnopyranosyl-(1-6)-β-D-glucopyranose], quercetin-3-O-[α-rhamnopyranosyl (1-4)α-L-rhamnopyranosyl (1-6) β-D-galactopyranoside] where R₁ is O-[α-rhamnopyranosyl (1-4)α-L-rhamnopyranosyl (1-6) β-D-galactopyranose], quercetin-3-O-[α-rhamnopyranosyl-(1-2)]-[β-glucopyranosyl-(1-6) ]-β-D-galactopyranoside where R₁ is O-(α-rhamnopyranosyl-(1-2)]-[β-glucopyranosyl-(1-6)]-β-D-galactopyranose, quercetin-3-O-[α-rhamnopyranosyl-(1-4)-α-rhamnopyranosyl-(1-6)-β-galactopyranoside] where R₁ is O-[α-rhamnopyranosyl-(1-4)-α-rhamnopyranosyl-(1-6)-β-galactopyranose], quercetin-3-O-α-L-rhamnopyranosyl-(1-2)-β-D-galactopyranoside where R₁ is O-α-L-rhamnopyranosyl-(1-2)-β-D-galactopyranose, quercetin-3-O-β-D-diglucopyranoside where R₁ is O-β-D-diglucopyranose, quercetin-3-O-β-D-galactoside-2″-gallate where R₁ is O-β-D-galactoside-2″-gallate, quercetin-3-O-β-D-glucopyranoside-(1-6)-β-D-galactopyranoside where R₁ is O-β-D-glucopyranoside-(1-6)-β-D-galactopyranose, quercetin-3-O-β-D-glucopyranosyl-(1-3)-α-L-rhamnopyranosyl-(1-6)-β-D-galactopyranoside where R₁ is O-β-D-glucopyranosyl-(1-3)-α-L-rhamnopyranosyl-(1-6)-β-D-galactopyranose, quercetin-3-O-β-D-glucuronide where R₁ is O-β-D-glucuronide, quercetin-3-O-β-D-xylopyranoside where R₁ is O-β-D-xylopyranose, quercetin-3-O-diglucospyranoside where R₁ is O-diglucospyranose, quercetin-3-O-gentiobioside where R₁ is O-gentiobiose, quercetin-3-O-glucopyranosylgalactopyranoside where R₁ is O-glucopyranosylgalactopyranose, quercetin-3-O-neohesperidoside where R₁ is O-neohesperidose, quercetin-3-O-sophoroside where R₁ is O-sophorose, quercetin-3-gentiotrioside where R₁ is gentiotriose, quercetin-3-methyl ether where R₁ is OCH₃, quercetin-3-rhamnogentiobioside where R₁ is rhamnogentiobiose, quercetin-3-rhamnoglucoside where R₁ is rhamnoglucose, and quercetin-3-sulfate where R₁ is sulfate; (ii) a derivative group of the formula I wherein R₁ is —OH, three functional groups out of R₂ to R₅ are —OH, and the rest one functional group varies, includes isorhamnetin where R₄ is OCH₃, quercimeritrin where R₃ is O-β-D-glucopyranose, rhamnetin where R₃ is OCH₃, quercetin-5-O-β-D-glucopyranoside where R₂ is O-β-D-glucopyranose, quercetin-7-O-β-D-glucuronopyranoside where R₃ is O-β-D-glucuronopyranose, and spireaoside where Rs is O-glucose; (iii) a derivative group of the formula I wherein three functional groups out of R₁ to Rs are OH and the rest two functional groups vary, includes rhamnazin where R₃ and R₄ are OCH₃, quercetin-3′,4′-di-methyl ether where R₄ and R₅ are OCH₃, quercetin-3,3′-dimethyl ether where R₁ and R₄ are OCH₃, quercetin-3,7-dimethyl ether where R₁ and R₃ are OCH₃, quercetin-3-O-[2″-O-(6′″-O-β-coumaroyl)-β-D-glucopyranosyl]-α-L-rhamnopyranosyl-7-O-β-D-glucopyranoside where R₁ is O-(2″-O-(6′″-O-β-coumaroyl)-β-D-glucopyranosyl]-α-L-rhamnopyranose and R₃ is O-β-D-glucopyranose, quercetin-3-O-[2″-O-61′-O-β-(7′″-O-β-D-glucopyranosyl)coumaroyl-β-D-glucopyranosyl]-α-L-rhamnopyranoside-7-O-β-D-glucopyranoside where R₁ is O-[2″-O-6′″-O-β-(7″″-O-β-D-glucopyranosyl)coumaroyl-β-D-glucopyranosyl]-α-L-rhamnopyranose and R₃ is O-β-D-glucopyranose, quercetin-3-O-rutinoside-7-O-β-D-glucopyranoside where R₁ is O-rutinose and R₃ is O-β-D-glucopyranose, quercetin-3-O-α-L-arabinopyranosyl-7-O-β-D-glucopyranoside where R₁ is O-α-L-arabinopyranosyl and R₃ is O-β-D-glucopyranose, quercetin-7-O-β-D-glucopyranoside-3-O-sophoroside where R₁ is O- sophorose and R₃ is O-β-D-glucopyranose, quercetin-3-O-galactopyranosyl-7-O-diglucopyranoside where R₁ is O-galactopyranose and R₃ is O-glucopyranose, quercetin-3-O-glucopyranosyl-7-diglucopyranoside where R₁ is O-glucopyranose and R₃ is O-glucopyranose, quercetin-3,7-diglucopyranoside where R₁ is glucopyranose and R₃ is glucopyranose, quercetin-3-gentiobiosyl-7-glucopyranoside where R₁ is gentiobiose and R₃ is glucopyranose, and quercetin-3, 4′-di-O-β-D-glucopyranoside where R₁ and R₅ are O-β-D-glucopyranose; and (iv) a derivative group of the formula I wherein more than three functional groups vary, includes quercetin-3,4′,7-trimethyl ether where R₁, R₃ and R₅ are OCH₃, and R₂ and R₄ are OH, and quercetin-3,3′,4′,7-tetramethyl ether where R₁, R₃, R₄ and R₅ are OCH₃, and R₂ is OH.

Quercetin having same OH groups in R₁ to R₅ of the above general formula (I) is a phenolic compound found in over 4000 kinds of plants in nature and is known as one of the phytoestrogens. It has a molecular formula of C₁₅H₁₀O₇ with resonance structures and a molecular weight of 302.33 g/mole and also known as vitamin P following the chemical structure identification in 1936. Quercetin is a rutin, a glycoside wherein sugar is linked via β-linkage and widely distributed in plants such as clover flower, pollen of common ragweed, and shell and stem of various plants, as well as in onion, kale, broccoli, lettuce, tomato, and apple. Quercetin has been verified not only to play an important role in maintenance of capillary wall integrity and capillary resistance (see: Gabor et al., Plant Flavonoids in Biology and Medicine II: Biochemical, Cellular, and Medical Properties, 280: 1-15, 19.88; Havsteen et al., Biochemical Pharmacology, 32:1141-1148, 1983) but also to have antioxidation activity, vitamin P activity, ultraviolet absorbing activity, antihypertensive activity, antiarrhythmic activity, antiinflamatory activity, antiallergic activity, anticholesteremic activity, suppressive activity on liver toxicity, and therapeutic effect on infertility, thus, it may be expected to use quercetin widely in foods, medical and pharmaceutical products, and cosmetics. However, there has been no report on the use of querceti.n for prevention and treatment of osteoporosis.

The therapeutic agent for osteoporosis of the invention comprising an active ingredient of quercetin derivative is illustrated below.

In order to search for the effects of quercetin derivatives on proliferation of osteoblasts and osteoclasts, the present inventors compared the effect of quercetin with that of phytoestrogen genistein which is known to be an effective agent for treatment of osteoporosis, and have found that quercetin has superior effects to genistein for activation of osteoblast proliferation, increase of alkaline phosphatase activity, and inhibition of osteoclast proliferation.

Furthermore, in ovariectomized rats, administration of quercetin derivatives has been found not to bring about changes in hormone level, proving that quercetin is a safe agent not causing uterine hypertrophy, an adverse side effect of estradiol which is being used as a therapeutic agent for osteoporosis currently. Also, quercetin derivatives were shown to be more effective than estradiol on increase of trabecular bone area of tibia which is apt to drastic change in trabecular bone area, and to have no adverse effect on hematopoietic function and immune system.

Therefore, quercetin derivatives of the invention, based on above results, have been found not only to have superior effects to currently using phytoestrogen genistein for activation of osteoblast proliferation and inhibition of osteoclast proliferation but also to have little side effects, bring about little change in hormone level and have no adverse effect on hematopoietic function and immune system, substantiating the use of quercetin derivatives as a therapeutic or preventive agent for osteoporosis.

Formulation

The said quercetin derivatives having superior effect on treatment of osteoporosis may be mixed with pharmaceutically acceptable excipients including binders such as polyvinylpyrrolidone, hydroxypropylcellulose, etc., disintegrating agents such as calcium carboxymethylcellulose, sodium glycolate starch, etc., diluting agents such as corn starch, lactose, soybean oil, crystalline cellulose, mannitol, etc., lubricating agents such as magnesium stearate, talc, etc., sweeteners such as sucrose, fructose, sorbitol, aspartame, etc., stabilizing agents such as sodium carboxymethylcellulose, α- or β-cyclodextrin, vitamin C, citric acid, white wax, etc, preservatives such as paraoxymethylbenzoate, paraoxypropylbenzoate, sodium benzoate, etc., and aromatics such as ethylvanillin, masking flavor, flavonomenthol, herb flavor, etc. to prepare pharmaceutical formulations for oral or parenteral administration such as tablets, capsules, soft capsules, liquids, ointments, pills, powders, suspensions, emulsions, syrups, suppositories or injections. Also, to augment efficacy of prevention and treatment of osteoporosis, calcium or vitamin D₃ may be added to the formulations. For parenteral administration of the pharmaceutical preparation of the invention, subcutaneous, intravenous, intramuscular or intraperitoneal injection may be employed. For parenteral administration, quercetin derivative may be mixed with stabilizer or buffer in water to prepare solution or suspension which can be produced as single-dose formulations of ampule or vial.

Dosage

The effective amount of quercetin in the therapeutic agent for osteoporosis of the invention is 2 to 20 mg/kg, preferably 8 to 12 mg/kg, which may be administered to the patient more than once a day depending on the patient's age, gender, degree of seriousness, way of administration, or purpose of prevention.

Safety

The toxicity of the quercetin derivatives of the invention has been reported in the literature (see: M. Sullivan et al., Proc. Soc. Exp. Biol. Med., 77:269, 1951) for the cases of oral administration and intraperitoneal administration to the mice, and LD₅₀ of orally administered quercetin was not less than 160 mg/kg, approving that quercetin is safe. In the present invention, liver, kidney, brain, uterus, skin and tibia were examined for the side effect of quercetin, which revealed that the weight of liver, kidney, brain, skin and tibia was not affected, moreover, uterine hypertrophy, a side effect of currently used therapeutic agents, was not observed with quercetin, proving that quercetin derivative as a hormone preparation can be used safely as a therapeutic agent for osteoporosis.

The present invention is further illustrated in the following examples, which should not be taken to limit the scope of the invention.

### EXAMPLE 1

### Effect of Quercetin on Osteoblast Proliferation

To analyse the effect of quercetin on osteoblast proliferation, human osteoblast-like cell line Saos-2 was employed and a phytoestrogen genistein was employed as a comparative agent which has been intensively studied as a therapeutic agent for osteoporosis.

### EXAMPLE 1-1

### Selection and Culture of Osteoblasts

Saos-2 cell line which has similar properties to osteoblasts was obtained from Korean Cell Line Bank affiliated to the Cancer Research Institute of School of Medicine, Seoul National University.

Saos-2 cells were seeded in a RPMI 1640 medium (Gibco BRL, U.S.A.) supplemented with 10% (v/v) FBS, 100 unit/ml penicillin, 100 gg/ml streptomycin and grown to form a monolayer in an incubator at 37° C. under an environment of 5% (v/v) CO₂ and saturated humidity. The culture was fed with fresh medium 2 to 3 times a week and subcultured once a week using 0.25% (w/v) trypsin.

### EXAMPLE 1-2

### Cell Proliferation Depending on Concentrations of the Agents

Saos-2 cells were distributed into a 96-well plate (20,000 cells/well) and quercetin in 1% DMSO was added to a final concentration of 10-2 to 10⁻⁹ mg/ml, 6 wells per each concentration. As a control group, cells without quercetin were used, and as a comparative group, the cells treated with various concentrations of genistein, being studied as a therapeutic agent for osteoporosis, were used. Cells were grown in an incubator at 37° C. for 3 days and incubated 4 more hours under the same condition after adding MTT (3-[4,5-dimethylthiazol-2-yl]-2,5-diphenyltetrazolium bromide, Triazolyl Blue) to a concentration of 0.05 mg/ml. Then, purple colored for-mazan formed in proportion to the number of viable cells was dissolved in DMSO and measured OD at 550 nm employing ELISA reader.

Cell proliferation rate (%) was evaluated by calculating the ratio of the OD of quercetin added well to the OD of control well, wherein, average value of ODs from 6 wells treated with the same concentration of quercetin was employed (see: Table 1).

cell proliferation rate (%)={(average value of OD at 550 nm of quercetin-treated wells—average value of OD at 550 nm of empty wells)/average value of OD at 550 nm of control wells}×100

### EXAMPLE 1-3

### Analysis of Alkaline Phosphatase (ALP) Activity

Since osteoblasts have cell specific alkaline phosphatase activity, the effect of quercetin of the invention on ALP activity in osteoblasts was evaluated as follows: the number of cells, concentration of tested agent, and culture condition were same as those used in MTT experiment of Example 1-2, and cells were harvested after 3 day-incubation. Genistein was used as a comparative agent. ALP activity was evaluated by analysing changes of OD at 405 nm result from hydrolysis of p-nitrophenylphosphate to p-nitrophenol and phosphate (see: Table 1).

TABLE 1
Effect of quercetin on osteoblast proliferation

| | Quercetin | | Genistein | |
|---|---|---|---|---|
| | (% of control group) | | (% of control group) | |
| Concentration (mg/ml) | MTT assay | ALP activity | MTT assay | ALP activity |
| Control | 100.0 ± 2.5 | 100.0 ± 1.6 | 100.0 ± 0.6 | 100.0 ± 7.3 |
| group | | | | |
| 1 × 10⁻⁹ | 93.1 ± 0.8* | 98.1 ± 0.0 | 91.3 ± 0.6* | 106.1 ± 6.4 |
| 1 × 10⁻⁸ | 93.9 ± 0.8 | 104.4 ± 3.9 | 96.9 ± 2.7 | 101.5 ± 8.8 |
| 1 × 10⁻⁷ | 98.6 ± 1.0 | 101.2 ± 3.1 | 95.9 ± 1.6 | 109.3 ± 9.6 |
| 1 × 10⁻⁶ | 96.0 ± 1.0 | 127.2 ± 3.5** | 90.5 ± 0.9** | 103.8 ± 8.7 |
| 1 × 10⁻⁵ | 95.8 ± 1.1 | 116.5 ± 3.7 | 97.3 ± 1.6 | 113.5 ± 7.3 |
| 1 × 10⁻⁴ | 96.5 ± 0.8 | 113.5 ± 2.3 | 95.7 ± 0.7 | 121.1 ± 6.2 |
| 1 × 10⁻³ | 98.3 ± 0.8 | 107.3 ± 1.5 | 85.5 ± 1.1** | 98.8 ± 6.9 |
| 1 × 10⁻² | 108.6 ± 2.2** | 106.1 ± 4.3 | 66.2 ± 2.8** | 62.3 ± 3.4 |

*p < 0.05
**p < 0.01

As shown in Table 1 above, in the cell proliferation experiment using MTT method, the cells treated with various concentrations of quercetin in the range of 1×10⁻⁹ to 1×10⁻³ mg/ml did not show any difference from the control cells which were not treated with the agent, while quercetin showed maximum cell proliferation effect of 109% of control cell proliferation at a concentration of 1×10⁻² mg/ml (p<0.01). On the other hand, genistein, a comparative agent, showed 91% (p<0.05) at a concentration of 1×10⁻⁹ mg/ml, 90.5% (p<0.01) at a concentration of 1×10⁻⁶ mg/ml, 86% (p<0.01) at a concentration of 1×10⁻³ mg/ml, and 66% (p<0.01) at a concentration of 1×10⁻²mg/ml, implying that genistein exert rather inhibitory effect than stimulatory effect on proliferation of osteoblasts.

In the experiment of assaying ALP activity, quercetin showed its maximum ALP activation effect of 127% (p<0.01) of control ALP activity at a concentration of 1×10⁻⁶ mg/ml, while genistein showed its maximum ALP activation activity of 121% at a concentration of 1×10⁻⁴ mg/ml, indicating that the ALP activation effect of quercetin of the invention is about 100 fold higher than that of genistein. Therefore, quercetin of the invention is more effective on the stimulation of osteoblast proliferation and activation of ALP activity than genistein which is studied intensively as a therapeutic agent for osteoporosis in recent years.

### EXAMPLE 2

### Effect of Quercetin on Osteoclast Proliferation

To examine whether quercetin have inhibitory effect on the proliferation of osteoclasts, experiments were carried out as followings.

### EXAMPLE 2-1

### Selection and Culture of Osteoclasts

ICR mice (Korea Research Institute of Chemical Technology, Taejon, Korea) were fed with calcium deficient diet (ICN Biomedicals, Inc., Ohio, U.S.A.) for 4 weeks to activate osteoclasts. The right and left tibiae and femurs of the calcium deficient rats were removed avoiding contamination of surrounding muscle tissues. Femurs and right and left tibiae, classified on the clean bench and kept on ice separately, were added into the α-MEM containing 100 μg/ml streptomycin and then vigorously shaken respectively to extract osteoclasts into the medium. After kept on ice for 5 minutes, the cell suspension was centrifuged at 800×g for 3 minutes and the cell pellet was resuspended in a α-MEM nutrient medium supplemented with 10% FBS, 100 μg/ml streptomycin and 100 unit/ml penicillin. The cell suspension was distributed into wells of a 24-well plate at a cell number of 3.5×10⁶/well.

### EXAMPLE 2-2

### Cell Proliferation Depending on Concentrations of Quercetin

To the osteoclasts obtained in Example 2-1 above, quercetin was added to yield concentrations of 1×10⁻⁸ to 1×10⁻² mg/ml. On day 2, the cells were subjected to tartrate-resistant acid phosphatase (TRAP) staining using a commercially available kit (Sigma Chemical Co., U.S.A.), followed by counting of osteoclasts which are TRAP-positive multinucleated cells (MNC), judged by more than three nuclei in a cell stained red (see: Table 2).

TABLE 2
Effect of quercetin on osteoclast proliferation

| Concentration | Number of osteoclast |
|---|---|
| (mg/ml) | (% of control group) |
| Control group | 100.0 ± 8.1 |
| 1 × 10⁻⁸ | 100.9 ± 1.8 |
| 1 × 10⁻⁶ | 96.8 ± 2.7 |
| 1 × 10⁻⁴ | 89.6 ± 3.2 |
| 1 × 10⁻³ | 61.1 ± 4.1* |
| 1 × 10⁻² | 24.7 ± 5.7** |

*p < 0.05,
**p < 0.01

As shown in Table 2 above, while quercetin at concentrations between 1×10⁻⁸ to 1×10⁻⁴ mg/ml exerted little inhibitory effect on the osteoclast proliferation, the cell numbers at quercetin concentration of 1×10⁻³ mg/ml and 1×10⁻² mg/ml was 61% (p<0.05%) and 25% of control cell number respectively, showing that quercetin exerted remarkable inhibitory effect on the osteoclast proliferation.

Based on the results of Examples 1 and 2, it was clearly demonstrated that quercetin is a potential therapeutic agent for osteoporosis which exerts stimulatory effect on osteoblast proliferation and inhibitory effect on osteoclast proliferation at a concentration of 10⁻² mg/ml.

### EXAMPLE 3

### Effect of Quercetin on Ovariectomized Rats

Female SD (Sprague-Dawley) rats, a model animal for type I osteoporosis occurring after menopause were employed for evaluating pharmacological effectiveness of quercetin. Female rats (10 weeks old) weighing 200 to 300 g, obtained from the Korea Research Institute of Chemical Technology were employed as experimental animals. Experiment was carried out by the procedure which comprises removing ovary, administration of agents to the each group of rats, and at certain days after ovariectomy, the rats were sacrificed and subjected to analyses including measurement of body weight, examination of internal organs, measurement of trabecular bone area, complete blood count, and biochemical analyses of plasma.

### EXAMPLE 3-1

### Ovariectomy and Administration of the Agents

Rats of control group and test group, except Sham group (normal group), were overiectomized as follows: a female rat was systemic anesthetized by intramuscular injection with 5 mg/100 g Ketamin (Yuhan Corporation, Korea) and 1 mg/100 g Xylazine (Beyer Korea, Korea) to the femur muscle of left and right hind limbs, and then, fur of lower abdominal region was shaved, operation area was sterilized with Potadin liquid (Iodine, Samil Pharm. Co., Ltd., Korea) in lying position, about 2 cm of abdominal skin, abdominal muscle, and peritoneum was cut in the middle under aseptic condition, ovary was exposed using sterilized forceps, followed by removal of both left and right ovaries after ligaturing of oviducts using silk threads. Subsequently, 0.3 ml of antibiotics (Sulfaforte®-4, Yoonee Chemical Co., Ltd., Korea) was. injected intraperitoneally to prevent infection, and then peritoneum, andominal muscle and skin were sutured with silk threads or nylon threads.

The Sham group, animals operated upon for the surgery as in the ovariectomized rats except for removing ovary, were employed to compare the changes caused solely by ovariectomy in control group which were ovariectomized but no agent was administered. Control group was employed to compare the changes caused by administration of agents in test group which were ovariectomized and administered with testing agents.

When test agents were administered, for a certain period of time before and after administration, 1.5 ml of blood was sampled from tail vein using a catheter (B.D Co.: 24 G) and subjected to complete blood count (Coulter Co.: JT) and biochemical analyses of plasma (Crone Co.: Airon® 200). During autopsy, blood was sampled from caudal venae cavae and subjected to the analyses above. And then, each sample was frozen to store for measurement of trabecular bone area of femur and examination of internal organs.

One week after operation, rats in Sham group and control group were intraperitoneally injected with 10% Tween 80 solution, the rats in E2 group were injected with 17β-estradiol at a concentration of 1 μg/kg/day, the rats in test group were injected with quercetin or genistein at a concentration of 10 mg/kg/day for 9 weeks, and the rats in each group were subjected to body weight measurement once a week. During the period of administration, blood was sampled once a week. After 9-week administration, entire blood was withdrawn with heparin treatment. Following complete blood count (CBC), the blood was centrifuged at 3,000 rpm for 20 minutes to obtain plasma which was stored at −70° C. until use. For measurement of bone mineral density, the lumbar spine L5 and L6, and right tibia were removed and stored separately in 4% (v/v) formalin solution.

### EXAMPLE 3-2

### Body Weight Change Depending on Quercetin Administration

The body weight of the rats in Sham group, E2 group treated with 17β-estradiol and test group treated with quercetin or genistein respectively, was measured once a week for 10 weeks after operation (see: Table 3).

TABLE 3
Mesurement of body weight changes depending on drug administration

| | Weight(g) | | | | |
|---|---|---|---|---|---|
| | | | | Quercetin- | Genistein- |
| Time | Control | | E2-treated | treated | treated |
| (week) | group | Sham group | group | group | group |
| Before | 219.39 ± 4.05 | 220.70 ± 4.63 | 228.51 ± 8.11 | 221.87 ± 7.57 | 217.55 ± 7.24 |
| operation | | | | | |
| 1 | 244.98 ± 3.00 | 231.51 ± 4.68 | 249.50 ± 8.16 | 241.73 ± 4.83 | 242.12 ± 5.96 |
| after | | | | | |
| operation | | | | | |
| 2 | 274.29 ± 3.68** | 236.40 ± 5.06^{##} | 264.97 ± 8.35 | 271.70 ± 5.79** | 270.00 ± 8.05** |
| after | | | | | |
| operation | | | | | |
| 3 | 299.37 ± 3.74** | 245.56 ± 4.79*^{##} | 279.87 ± 8.15** | 295.00 ± 3.89** | 296.20 ± 7.68** |
| after | | | | | |
| operation | | | | | |
| 4 | 315.20 ± 3.84** | 248.96 ± 5.02*^{##} | 292.83 ± 9.25** | 312.07 ± 5.95** | 310.80 ± 7.80** |
| after | | | | | |
| operation | | | | | |
| 5 | 320.30 ± 4.83** | 255.43 ± 5.14**^{##} | 296.96 ± 9.44** | 320.25 ± 6.76** | 317.29 ± 7.93** |
| after | | | | | |
| operation | | | | | |
| 6 | 329.03 ± 5.05** | 261.49 ± 6.46**^{##} | 304.49 ± 8.40** | 326.68 ± 6.73** | 327.19 ± 8.31** |
| after | | | | | |
| operation | | | | | |
| 7 | 337.39 ± 5.93** | 264.78 ± 5.53**^{##} | 313.04 ± 8.73** | 333.25 ± 7.61** | 332.80 ± 9.23** |
| after | | | | | |
| operation | | | | | |
| 8 | 340.01 ± 6.60** | 268.16 ± 5.40**^{##} | 315.87 ± 8.32** | 335.09 ± 6.65** | 336.38 ± 9.01** |
| after | | | | | |
| operation | | | | | |
| 9 | 347.96 ± 7.58** | 273.81 ± 4.54**^{##} | 319.95 ± 9.47** | 343.02 ± 6.96** | 342.71 ± 8.26** |
| after | | | | | |
| operation | | | | | |
| 10 | 356.73 ± 7.13** | 275.22 ± 4.30**^{##} | 320.00 ± 5.90**^{#} | 346.27 ± 6.39** | 347.23 ± 7.57** |
| after | | | | | |
| operation | | | | | |

*p < 0.05,
**p < 0.01, compared with before operation
^{#}p < 0.05,
^{##}p < 0.01, compared with control group

As shown in Table 3, body weight of Sham group began to increase 3 weeks (p<0.05) after operation and that of control group began to increase 2 weeks (p<0.01) after operation. That is, control group showed rapid increase of body weight compare to Sham group, and such increase of body weight was slowed down after administration of estradiol, and E2 group showed slower increase of body weight compare to control group (p<0.05) 20 weeks after operation. Meanwhile, the test group administered with phytoestrogen quercetin or genistein at a concentration of 10 mg/kg/day respectively showed rapid increase of body weight even after removing ovary similar to control group. Thus, quercetin administration was found not to bring about meaningful changes in hormone level in the body.

### EXAMPLE 3-3

### Changes in the Weight of Internal Organ by Quercetin

To find out quercetin effect on internal organ of test animal, liver, kidney, brain, uterus, skin, and tibia were removed from the test animals administered with test agents for 9 weeks after operation and wet weight of each organ was measured (see: Table 4).

TABLE 4
Changes in the weight of internal organ after drug administeration

| | | | E2- | Quercetin- | Genistein- |
|---|---|---|---|---|---|
| | Control | Sham | treated | treated | treated |
| | group | group | group | group | group |
| Liver | 9.84 ± 0.33 | 9.52 ± 0.48 | 9.22 ± 0.43 | 9.07 ± 0.30 | 10.03 ± 0.36 |
| (g) | | | | | |
| Kidney | 1.95 ± 0.09 | 1.91 ± 0.05 | 1.85 ± 0.09 | 1.84 ± 0.05 | 1.83 ± 0.03 |
| (g) | | | | | |
| Brain | 2.03 ± 0.04 | 1.93 ± 0.02 | 1.98 ± 0.05 | 1.98 ± 0.04 | 1.98 ± 0.03 |
| (g) | | | | | |
| Tibia | 0.559 ± 0.025 | 0.514 ± 0.013 | 0.504 ± 0.019 | 0.554 ± 0.019 | 0.537 ± 0.008 |
| (g) | | | | | |
| Skin | 193 ± 7 | 169 ± 8 | 193 ± 6 | 197 ± 11 | 188 ± 9 |
| (mg) | | | | | |
| Uterus | 79 ± 4 | 450 ± 29** | 279 ± 10** | 85 ± 6 | 106 ± 3 |
| (mg) | | | | | |

**p < 0.01

As shown in Table 4, in case of the weight of liver, kidney, brain, tibia, and skin, normal Sham group, ovariectomized control group and test group did not show differences among groups. However, in case of weight of uterus which is affected by the estrogen secreted from ovary, ovariectomized control group showed significant decrease (p<0.01) compare to Sham group, and administration of E2 after removing ovary suppressed atrophy of uterus (p<0.01) compare to control group. Administration of phytoestrogen quercetin or genistein did not give rise to change in weight of uterus, on the other hand, E2 which is a currently used therapeutic agent for osteoporosis showed side effect such as uterine hypertrophy, showing that quercetin can be used safely as a therapeutic agent for osteoporosis without adverse side effect.

### EXAMPLE 3-4

### Changes in the Trabecular Bone Area By Quercetin

Trabecular bone area (TBA) of lumbar and tibia removed from the rats of each group which was treated with various agents for 9 weeks were measured as follows: that is, using a digitalizer of quantitative image analysis system (Wild Leitz Co.), image of each trabecula was obtained on computer monitor by drawing a contour of the trabecula, and then, using a computer, calculated were average areas of trabeculae within a rectangle of 2×10⁶ μm² area wherein the width is about ⅔ of the length of growth plate which located underneath of growth plate at proximity of tibia. Also, following the number of trabeculae within the rectangle were obtained, average area was multiplied by the number of trabeculae to obtain trabecular bone area of each sample bone, which was analyzed statistically (see: Table 5).

TABLE 5
Changes in the trabecular bone area of tibia
depending on drug administration

| | TBA (×10⁴ μm²) | Change Rate (%) |
|---|---|---|
| Control group | 34.62 ± 2.62 | 100.00 ± 7.55 |
| Sham group | 85.55 ± 5.31** | 247.07 ± 15.33** |
| E2-treated | 51.40 ± 2.28 | 148.46 ± 6.59 |
| group | | |
| Quercetin- | 55.52 ± 7.68* | 160.34 ± 22.17* |
| treated group | | |
| Genistein- | 47.65 ± 2.07 | 137.62 ± 5.98 |
| treated group | | |

*p < 0.05,
**p < 0.01

As shown in Table 5, in case of tibia, the TBA of control group was 34.62×10⁻⁴ μm² which is a significantly decreased value compare to normal Sham group of 85.55×10⁴ μm² (p<0.01), showing that osteoporosis have occurred in control group, and such decreased TBA was increased again by treatment with E2, quercetin or genistein to 148%, 160%, and 138% of TBA of control group respectively, especially in case of quercetin, remarkable increase of TBA was monitored (p<0.05).

TBAs of lumbars removed from the animal treated with test agents for 9 weeks were measured employing the same method above (see: Table 6).

TABLE 6
Changes in the trabecular bone area of lumbars
depending on drug administration

| | TBA (×10⁴ μm²) | Change Rate (%) |
|---|---|---|
| Control group | 67.53 ± 2.31 | 100.00 ± 3.42 |
| Sham group | 93.70 ± 5.29** | 138.76 ± 7.84** |
| E2-treated group | 89.16 ± 2.83** | 132.04 ± 4.19** |
| Quercetin-treated | 87.38 ± 4.53* | 129.40 ± 6.71* |
| group | | |
| Genistein-treated | 86.58 ± 3.00* | 128.23 ± 4.45* |
| group | | |

*p < 0.05,
**p < 0.01

As shown in Table 6, in case of lumbar, the TBA of control group was 67.53×10⁴ μm² which is a decreased value compare to Sham group of 93.7×10⁴ μm² (p<0.01), but, such decreased TBA was increased again by treatment with E2, quercetin or genistein to 132% (p<0.01), 129% (p<0.05) and 128% (p<0.05) of TBA of control group respectively, showing that these test agents exerted suppressing effect on decrease of TBA caused by ovariectomy. Especially, quercetin showed more significant increase of TBA in tibia which is apt to drastic change in TBA than E2 a currently used therapeutic agent for osteoporosis, showing that quercetin is a more effective therapeutic agent not causing uterine hypertrophy which is an adverse side effect caused by E2.

### EXAMPLE 3-5

### Complete Blood Count

Complete blood count which reflects the condition and abnormality of the body was measured to find out abnormality in test animals caused by administrtion of agents. That is, to find out changes in hematopoiesis of test rats, measured were red blood cell (RBC) count, concentration of hemoglobin (Hb) and hematocrit (Ht) of-blood samples obtained from the rats prior to operation and the rats 10 weeks after administrating agents following operation, and to find out changes in immune system such as inflammation and necrosis of tissues, measured were white blood cell count, lymphocyte count, monocyte count, and granulocyte count (see: Table 7).

TABLE 7
Changes in Complete blood count depending on drug administration

| | | | | E2- | Quercetin- | Genistein- |
|---|---|---|---|---|---|---|
| | | Control | Sham | treated | treated | treated |
| | Operation | group | group | group | group | group |
| Red blood | before | 7.36 ± 0.11 | 7.19 ± 0.11 | 7.33 ± 0.13 | 7.29 ± 0.15 | 7.32 ± 0.13 |
| cell(RBC) count | after | 7.08 ± 0.09 | 6.75 ± 0.24 | 6.97 ± 0.14 | 7.13 ± 0.15 | 7.17 ± 0.13 |
| (×10⁶ cells/μl) | | | | | | |
| Concentration | before | 16.09 ± 0.21 | 15.75 ± 0.20 | 15.86 ± 0.24 | 16.00 ± 0.30 | 15.82 ± 0.27 |
| of | | | | | | |
| hemoglobin(Hb) | after | 14.58 ± 0.20** | 14.09 ± 0.48** | 14.34 ± 0.29** | 14.84 ± 0.22* | 14.70 ± 0.22** |
| (g/dl) | | | | | | |
| Hematocrit(Ht) | before | 43.34 ± 0.48 | 43.09 ± 0.61 | 43.11 ± 0.55 | 43.62 ± 0.83 | 42.76 ± 0.65 |
| (%) | after | 39.48 ± 0.60** | 38.39 ± 1.24** | 38.86 ± 0.72** | 41.10 ± 0.68* | 40.66 ± 0.56* |
| White blood | before | 26.13 ± 4.63 | 25.61 ± 3.64 | 23.14 ± 1.50 | 20.28 ± 3.77 | 27.30 ± 4.85 |
| cell count | after | 21.66 ± 2.89 | 12.74 ± 2.88* | 13.26 ± 0.97** | 18.50 ± 7.60 | 21.50 ± 2.53 |
| (×10³ cells/μl) | | | | | | |
| Lymphocyte | before | 22.14 ± 4.49 | 18.04 ± 2.38 | 17.80 ± 1.72 | 16.78 ± 3.52 | 19.68 ± 4.52 |
| count | after | 21.20 ± 9.00 | 10.20 ± 2.88 | 10.23 ± 0.96** | 15.00 ± 7.71 | 15.25 ± 3.21 |
| (×10³ cells/μl) | | | | | | |
| Monocyte count | before | 1.02 ± 0.18 | 0.73 ± 0.17 | 1.44 ± 0.29 | 0.65 ± 0.07 | 0.77 ± 0.09 |
| (×10³ cells/μl) | after | 1.10 ± 0.21 | 0.95 ± 0.14 | 1.02 ± 0.24 | 1.00 ± 0.20 | 0.80 ± 0.19 |
| Granulocyte | before | 2.99 ± 0.44 | 2.83 ± 0.39 | 3.67 ± 0.40 | 2.80 ± 0.30 | 2.23 ± 0.10 |
| count | after | 2.52 ± 0.21 | 1.93 ± 0.26 | 1.99 ± 0.25** | 2.43 ± 0.12 | 2.38 ± 0.37 |
| (×10³ cells/μl) | | | | | | |

*p < 0.05,
**p < 0.01

As shown in Table 7, RBC count did not show any changes before and after operation in all groups, and concentration of hemoglobin and hematocrit were decreased after operation in all groups. White blood cell count did not show any changes before and after operation in quercetin or genistein treated groups, but decreased in Sham group and E2 group after operation. Also, lymphocyte and granulocyte count showed rapid decrease in E2 group only, and momocyte count was stayed same in entire groups. Thus, quercetin was found to be a safe agent not disturbing hematopoiesis and immune system of the body.

### EXAMPLE 3-6

### Biochemical Changes of Plasma by Quercetin

Since blood reflects the condition of body, safety of quercetin in the body was evaluated by measuring biochemical parameters: that is, blood samples were obtained from the rat prior to operation, one week after operation, and 10 weeks after operation, and measured were levels of alkaline phosphatase (ALP), calcium, inorganic phosphate, blood urea nitrogen (BUN), creatinin, total cholesterol, HDL-cholesterol and LDL-cholesterol (see: Table 8).

TABLE 8
Changes in biochemical parameters in plasma depending on drug administration

| | | | | E2- | Quercetin- | Genistein- |
|---|---|---|---|---|---|---|
| | | Control | Sham | treated | treated | treated |
| | Operation | group | group | group | group | group |
| Concentration | before | 262.75 ± 23.31 | 245.59 ± 22.05 | 196.01 ± 28.34 | 232.83 ± 20.27 | 208.86 ± 19.72 |
| of ALP | 1 week | 265.75 ± 22.78 | 215.18 ± 20.22 | 195.24 ± 27.87 | 226.67 ± 23.20 | 212.10 ± 17.92 |
| (U/dL) | after | | | | | |
| | 10 | 198.31 ± 14.64 | 135.09 ± 18.64^{##$} | 123.99 ± 22.18 | 156.42 ± 13.08 | 127.14 ± 9.95^{##$$} |
| | weeks | | | | | |
| | after | | | | | |
| Concentration | before | 10.48 ± 0.43 | 10.57 ± 0.55 | 10.86 ± 0.40 | 10.73 ± 0.48 | 10.61 ± 0.49 |
| of | 1 week | 9.98 ± 0.34 | 10.35 ± 0.17 | 10.03 ± 0.18 | 8.37 ± 0.24**# | 8.97 ± 0.29^{#} |
| calcium | after | | | | | |
| (mg/dL) | 10 | 10.83 ± 0.16 | 11.79 ± 0.23*$ | 11.20 ± 0.16^{$} | 10.26 ± 0.19^{$} | 10.44 ± 0.22^{$} |
| | weeks | | | | | |
| | after | | | | | |
| Concentration | before | 6.52 ± 0.39 | 6.87 ± 0.62 | 6.90 ± 0.52 | 6.79 ± 0.66 | 7.18 ± 0.48 |
| of | 1 week | 6.27 ± 0.31 | 6.59 ± 0.20 | 6.13 ± 0.12 | 6.21 ± 0.18 | 6.47 ± 0.16 |
| inorganic | after | | | | | |
| phosphate | 10 | 4.95 ± 0.41^{##} | 6.09 ± 0.47 | 5.51 ± 0.45 | 5.73 ± 0.58 | 5.62 ± 0.25^{#} |
| (mg/dL) | weeks | | | | | |
| | after | | | | | |
| Concentration | before | 18.56 ± 0.92 | 17.13 ± 1.11 | 18.36 ± 1.01 | 17.05 ± 0.60 | 16.82 ± 0.60 |
| of | 1 week | 18.31 ± 0.70 | 16.75 ± 0.58 | 17.79 ± 0.76 | 18.06 ± 0.88 | 18.26 ± 0.94 |
| blood | after | | | | | |
| urea | 10 | 21.20 ± 1.06 | 19.23 ± 0.84 | 19.99 ± 0.86 | 18.19 ± 0.41 | 18.31 ± 0.86 |
| nitrogen | weeks | | | | | |
| (BUN) | after | | | | | |
| (mg/dL) | | | | | | |
| Concentration | before | 0.54 ± 0.05 | 0.56 ± 0.06 | 0.55 ± 0.05 | 0.57 ± 0.05 | 0.51 ± 0.04 |
| of | 1 week | 0.54 ± 0.05 | 0.62 ± 0.04 | 0.57 ± 0.03 | 0.59 ± 0.01 | 0.64 ± 0.02* |
| creatin | after | | | | | |
| in | 10 | 0.78 ± 0.03^{##$$} | 0.80 ± 0.03^{##} | 0.81 ± 0.03^{##$$} | 0.82 ± 0.04^{##$} | 0.82 ± 0.04^{##$} |
| (mg/dL) | weeks | | | | | |
| | after | | | | | |
| Concentration | before | 72.66 ± 5.00 | 79.67 ± 1.73 | 76.79 ± 2.80 | 77.55 ± 5.13 | 85.51 ± 5.45 |
| of | 1 week | 93.32 ± 4.75^{#} | 79.75 ± 2.46 | 95.53 ± 4.17 | 85.84 ± 3.82 | 91.56 ± 3.65 |
| total | after | | | | | |
| cholesterol | 10 | 120.44 ± 5.21^{##$$} | 88.60 ± 4.87**# | 115.05 ± 5.75^{##$} | 107.73 ± 2.24^{##} | 121.07 ± 6.53^{##} |
| (mg/dL) | weeks | | | | | |
| | after | | | | | |
| Concentration | before | 53.78 ± 2.77 | 52.33 ± 2.61 | 52.30 ± 2.01 | 53.38 ± 3.14 | 61.12 ± 3.57 |
| of HDL- | 1 week | 46.20 ± 0.62 | 41.69 ± 1.47 | 49.03 ± 3.37 | 42.49 ± 4.85 | 35.26 ± 1.92^{##} |
| cholesterol | after | | | | | |
| (mg/dL) | 10 | 29.60 ± 2.63^{##$$} | 22.32 ± 2.49^{##$$} | 24.94 ± 2.72^{##$$} | 25.13 ± 2.78^{##} | 29.27 ± 1.98^{##} |
| | weeks | | | | | |
| | after | | | | | |
| Concentration | before | 18.88 ± 3.15 | 26.63 ± 3.04 | 24.49 ± 1.63 | 24.17 ± 3.13 | 24.39 ± 3.63 |
| of LDL- | 1 week | 42.80 ± 6.41^{##} | 36.30 ± 0.63 | 40.50 ± 6.17 | 40.85 ± 4.88 | 60.47 ± 7.04^{##} |
| cholesterol | after | | | | | |
| (mg/dL) | 10 | 90.84 ± 4.27^{##$$} | 69.29 ± 3.05^{##$$} | 88.33 ± 4.74^{##$$} | 82.60 ± 4.85^{##$$} | 91.80 ± 6.57^{##$$} |
| | weeks | | | | | |
| | after | | | | | |

*p < 0.05,
**p < 0.01, compared with control group
^{#}p < 0.05,
^{##}p < 0.01, compared with before operation
^{$}p < 0.05,
^{$$}p < 0.01, compared with 1 week after operation

As shown in Table 8, ALP activity which is directly related to bone metabolism showed tendency of decrease with aging in entire groups, especially, in Sham group and genistein treated group, the rats of 10 weeks after operation showed significant decrease of ALP activity and no change in calcium concentration compare to the rats prior to operation and one week after operation. And, the level of inorganic phosphate remarkably decreased in the rats of 10 weeks after operation compare to the rats prior to operation in control group and genistein treated group.

While the level of blood urea nitrogen which is related to the protein metabolism and muscle volume was maintained at a proper level in entire groups, the level of creatinin increased in entire groups.

The level of total cholesterol which is known to increase in postmenopause women increased in entire groups, although increase in Sham group was relatively low. While the level of HDL-cholesterol decreased with time in entire groups, the level of LDL-cholesterol increased with time, which were found in normal Sham group as well as ovariectomized groups.

Thus, the quercetin of the invention was found to be an effective therapeutic and preventive agent for osteoporosis.

### EXAMPLE 4

### The Formulation of the Quercetin Preparation

### EXAMPLE 4-1

### Syrup

The syrup formulation containing 2% (w/v) quercetin, its derivatives or pharmaceutically acceptable salts thereof was prepared as follows: quercetin hydrochloride, saccharine and sugar were dissolved in 80 g of warm water, cooled down, and then mixed with a solution containing glycerin, saccharine, aromatics, ethanol, sorbic acid and distilled water. Water was added to the mixture prepared above to give 100 ml of syrup formulation of quercetin, whose components are as follows:

| | | |
|---|---|---|
| quercetin hydrochloride | 2 | g |
| saccharine | 0.8 | g |
| sugar | 25.4 | g |
| glycerin | 8.0 | g |
| aromatics | 0.04 | g |
| ethanol | 4.0 | g |
| sorbic acid | 0.4 | g |

| | |
|---|---|
| distilled water | a proper quantity |

### EXAMPLE 4-2

### Tablet

The tablet containing quercetin, its derivatives or pharmaceutically acceptable salts thereof was prepared as follows: 250 g of flavonoid derivative of quercetin hydrochloride was mixed with 175.9 g of lactose, 180 g of potato starch, and 32 g of colloidal silicate, and then 10% (w/v) gelatin solution was added. After pulverization, the mixture was passed through a 14-mesh sieve, dried, and mixed with 160 g of potato starch, 50 g of talc, and 5 g of magnesium stearate to give tablets, whose components are as follows:

| | |
|---|---|
| flavonoid derivative of quercetin hydrochloride | 250 g |
| lactose | 175.9 g |
| potato starch | 180 g |
| colloidal silicate | 32 g |
| 10% (w/v) gelatin solution | a proper quantity |
| potato starch | 160 g |
| talc | 50 g |
| magnesium stearate | 5 g |

### EXAMPLE 4-3

### Injection

One gram of flavonoid derivative of quercetin hydrochloride, 0.6 g NaCl, and 0.1 g of ascorbic acid were dissolved in distilled water to give a final volume of 100 ml, and then the solution was put into a vial, which was sterilized by heating at 100° C. for 30 minutes to give the injection. The components of the said injection are as follows:

| | |
|---|---|
| flavonoid derivative of quercetin hydrochloride | 1 g |
| NaCl | 0.6 g |
| ascorbic acid | 0.1 g |
| distilled water | a proper quantity |

As clearly illustrated and demonstrated above, the present invention provides a therapeutic agent for osteoporosis comprising an active ingredient of quercetin derivatives which effectively stimulate osteoblast proliferation and inhibit osteoclast proliferation. The quercetin derivatives of the invention can be practically applied for the treatment and prevention of osteoporosis, since they effectively inhibit osteoclast proliferation and stimulate osteoblast proliferation more than conventional therapeutic agents for osteoporosis, and increase trabecular bone area highly without changing hormone level in body and untoward effects on hematopoietic function and immune system.

What is claimed is:

1. A method of ameliorating the symptoms associated with osteoporosis, said method comprising:
administering to said subject a therapeutic agent comprising quercetin or at least one derivative thereof represented by the following general formula (I) 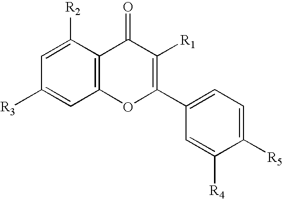
wherein,
R₁ is gentiotriose, glucopyranose, O-arabinofuranose, O-diglucopyranose, O-galactopyranose, O-galactoside-gallate, O-gentiobiose, O-glucopyranose, O-glucuronide, O-neohesperidose, O-rhamnopyranose, O-sophorose, O-xylopyranose, OCH₃, OH, rhamnogentiobiose, rhamnoglucose, or sulfate;
R₂ is OH or O-glucopyranose;
R₃ is OCH₃, OH, O-glucopyranose, O-glucuronopyranose or glucopyranose;
R₄ is OCH₃, OH; and
R₅ is OCH₃, OH, O-glucopyranose or O-glucose, and wherein said therapeutic agent lacks calcium.

2. The method of claim 1, wherein said quercetin or at least one derivative thereof is selected from the group consisting of quercetin, avicularoside, guiajaverin, hyperoside, isohyperoside, isoquercitrin, multinoside A, multinoside A acetate, quercitrin, quercetin-3-O-(2″-O-β-D-glucopyranosyl)-α-L-rhamnopyranoside, quercetin-3-O-(6″-O-galloyl)-glucopyranoside, quercetin-3-O-(6′″-O-p-coumaroyl-β-D-glucopyranosyl-(1-2)-α-L-rhamnopyrano side), quercetin-3-O-D-glucopyranosyl-(1-6)-β-D-glucopyranosyl-(1-4)-α-L-rhamnopyranoside, quercetin-3-O-[2″-O-6′″-O-p-(7″″-O-β-D-glucopyranosyl)coumaroyl-β-D-glucopyranosyl]-α-L-rhamnopyranoside, quercetin-3-O-[6′″-p-coumaroyl-β-D-glucopyranosyl-β-(1-4)-rhamnopyranoside], quercetin-3-O-[α-L-rhamnopyranosyl (1-2)-α-L-rhamnopyranosyl-(1-6)-β-D-glucopyranoside], quercetin-3-O-[α-rhamnopyranosyl (1-4)α-L-rhamnopyranosyl (1-6)β-D-galactopyranoside], quercetin-3-O-[α-rhamnopyranosyl-(1-2)]-[β-glucopyranosyl-(1-6)]-β-D-galactopyranoside, quercetin-3-O-[α-rhamnopyranosyl-(1-4)-α-rhamnopyranosyl-(1-6)-β-galactopyrano side], quercetin-3-O-α-L-rhamnopyranosyl-(1-2)-β-D-galactopyranoside, quercetin-3-O-β-D-diglucopyranoside, quercetin-3-O-β-D-galactoside-2″-gallate, quercetin-3-O-β-D-glucopyranoside-(1-6)-β-D-galactopyranoside, quercetin-3-O-β-D-glucopyranosyl-(1-3)-α-L-rhamnopyranosyl-(1-6)-β-D-galactopyranoside, quercetin-3-O-β-D-glucuronide, quercetin-3-O-β-D-xylopyranoside, quercetin-3-O-diglucospyranoside, quercetin-3-O-gentiobioside, quercetin-3-O-glucopyranosylgalactopyranoside, quercetin-3-O-neohesperidoside, quercetin-3-gentiotrioside, quercetin-3-methyl ether, quercetin-3-rhamnogentiobioside, quercetin-3-rhamnoglucoside and quercetin-3-sulfate.

3. The method of claim 1, wherein said at least one quercetin derivative is selected from the group consisting of isorhamnetin, quercimeritrin, rhamnetin, quercetin-5-O-β-D-glucopyranoside, quercetin-7-O-β-D-glucuronopyranoside and spireaoside.

4. The method of claim 1, wherein said at least one quercetin derivative is selected from the group consisting of rhamnazin, quercetin-3′,4′-di-methyl ether, quercetin-3,3′-dimethyl ether, quercetin-3,7-dimethyl ether, quercetin-3-O-[2″-O-(6′″-O-β-coumaroyl)-β-D-glucopyranosyl]-α-L-rhamnopyranosyl-7-O-β-D-glucopyranoside, quercetin-3-O-[2″-O-6′″-O-β-(7″″-O-β-D-glucopyranosyl)coumaroyl-α-D-glucopranosyl]-α-L-rharnnopyranoside-7-O-β-D-glucopyranoside, quercetin-3-O-rutinoside-7-O-β-D-glucopyranoside, quercetin-3-O-α-L-arabinopyranosyl-7-O-β-D-glucopyranoside, quercetin-7-O-β-D-glucopyranoside-3-O-sophoroside, quercetin-3-O-galactopyranosyl-7-O-diglucopyranoside, quercetin-3-O-glucopyranosyl-7-diglucopyranoside, quercetin-3,7-diglucopyranoside, quercetin-3-gentiobiosyl-7-glucopyranoside and quercetin-3,4′-di-O-β-D-glucopyranoside.

5. The method of claim 1, wherein said at least one quercetin derivative is selected from the group consisting of quercetin-3,4′,7-trimethyl ether and quercetin-3,3′,4′,7-tetramethyl ether.

6. A method of ameliorating the symptoms associated with osteoporosis, said method comprising:
administering to said subject a therapeutic agent consisting essentially of quercetin or at least one derivative thereof represented by the following general formula (I) 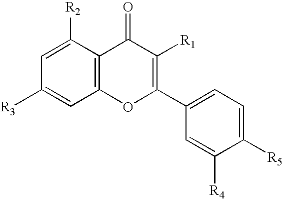
wherein,
R₁ is gentiotriose, glucopyranose, O-arabinofuranose, O-diglucopyranose, O-galactopyranose, O-galactoside-gallate, O-gentiobiose, O-glucopyranose, O-glucuronide, O-neohesperidose, O-rhamnopyranose, O-sophorose, O-xylopyranose, OCH₃, OH, rhamnogentiobiose, rhamnoglucose, or sulfate;
R₂ is OH or O-glucopyranose;
R₃ is OCH₃, OH, O-glucopyranose, O-glucuronopyranose or glucopyranose;
R₄ is OCH₃, OH; and
R₅ is OCH₃, OH, O-glucopyranose or O-glucose.

7. The method of claim 6, wherein said quercetin or at least one derivative thereof is selected from the group consisting of quercetin, avicularoside, guiajaverin, hyperoside, isohyperoside, isoquercitrin, multinoside A, multinoside A acetate, quercitrin, quercetin-3-O-(2″-O-β-D-glucopyranosyl)-α-L-rhamnopyranoside, quercetin-3-O-(6″-O-galloyl)-glucopyranoside, quercetin-3-O-(6′″-O-β-coumaroyl-β-D-glucopyranosyl-(1-2)-α-L-rhamnopyrano side), quercetin-3-O-D-glucopyranosyl-(1-6)-β-D-glucopyranosyl-(1-4)-α-L-rhamnopyranoside, quercetin-3-O-[2″-O-6′″-O-β-(7″″-O-O-D-glucopyranosyl)coumaroyl-β-D-glucopyranosyl]-α-L-rhamnopyranoside, quercetin-3-O-[6′″-β-coumaroyl-β-D-glucopyranosyl-β-(1-4)-rhamnopyranoside], quercetin-3-O-[α-L-rhamnopyranosyl (1-2)-α-L-rhamnopyranosyl-(1-6)-β-D-glucopyranoside], quercetin-3-O-[α-rhamnopyranosyl (1-4)α-L-rhamnopyranosyl (1-6)β-D-galactopyranoside], quercetin-3-O-[α-rhamnopyranosyl-(1-2)]-[β-glucopyranosyl-(1-6)]-β-D-galactopyranoside, quercetin-3-O-[α-rhamnopyranosyl-(1-4)-α-rhamnopyranosyl-(1-6)-β-galactopyranoside], quercetin-3-O-α-L-rhamnopyranosyl-(1-2)-β-D-galactopyranoside, quercetin-3-O-β-D-diglucopyranoside, quercetin-3-O-β-D-galactoside-2″-gallate, quercetin-3-O-β-D-glucopyranoside-(1-6)-β-D-galactopyranoside, quercetin-3-O-β-D-glucopyranosyl-(1-3)-α-L-rhamnopyranosyl-(1-6)-β-D-galactopyranoside, quercetin-3-O-β-D-glucuronide, quercetin-3-O-β-D-xylopyranoside, quercetin-3-O-diglucospyranoside, quercetin-3-O-gentiobioside, quercetin-3-O-glucopyranosylgalactopyranoside, quercetin-3-O-neohesperidoside, quercetin-3-gentiotrioside, quercetin-3-methyl ether, quercetin-3-rhamnogentiobioside, quercetin-3-rhamnoglucoside and quercetin-3-sulfate.

8. The method of claim 6, wherein said at least one quercetin derivative is selected from the group consisting of isorhamnetin, quercimeritrin, rhamnetin, quercetin-5-O-β-D-glucopyranoside, quercetin-7-O-β-D-glucuronopyranoside and spireaoside.

9. The method of claim 6, wherein said at least one quercetin derivative is selected from the group consisting of rhamnazin, quercetin-3′,4′-di-methyl ether, quercetin-3,3′-dimethyl ether, quercetin-3,7-dimethyl ether, quercetin-3-O-[2″-O-(6′″-O-p-coumaroyl)-β-D-glucopyranosyl]-α-L-rhamnopyranosyl-7-O-β-D-glucopyranoside, quercetin-3-O-[2″-O-6′″-O-β-(7″″-O-13-D-glucopyranosyl)coumaroyl-β-D-glucopranosyl]-α-L-rhamnopyranoside-7-O-β-D-glucopyranoside, quercetin-3-O-rutinoside-7-O-β-D-glucopyranoside, quercetin-3-O-α-L-arabinopyranosyl-7-O-β-D-glucopyranoside, quercetin-7-O-β-D-glucopyranoside-3-O-sophoroside, quercetin-3-O-galactopyranosyl-7-O-diglucopyranoside, quercetin-3-O-glucopyranosyl-7-diglucopyranoside, quercetin-3,7-diglucopyranoside, quercetin-3-gentiobiosyl-7-glucopyranoside and quercetin-3,4′-di-O-β-D-glucopyranoside.

10. The method of claim 6, wherein said at least one quercetin derivative is selected from the group consisting of quercetin-3,4′,7-trimethyl ether and quercetin-3,3′,4′,7-tetramethyl ether.

11. A method of ameliorating the symptoms associated with osteoporosis, said method comprising:
increasing the trabecular bone area of a subject by administering to said subject a therapeutic agent, which comprises quercetin or at least one derivative thereof represented by the following general formula (I) 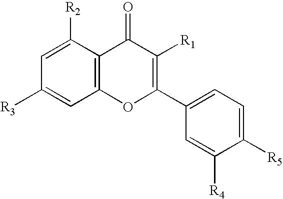
wherein,
R₁ is gentiotriose, glucopyranose, O-arabinofuranose, O-diglucopyranose, O-galactopyranose, O-galactoside-gallate, O-gentiobiose, O-glucopyranose, O-glucuronide, O-neohesperidose, O-rhamnopyranose, O-sophorose, O-xylopyranose, OCH₃, OH, rhamnogentiobiose, rhamnoglucose, or sulfate;
R₂ is OH or O-glucopyranose;
R₃ is OCH₃, OH, O-glucopyranose, O-glucuronopyranose or glucopyranose;
R₄ is OCH₃, OH; and
R₅ is OCH₃, OH, O-glucopyranose or O-glucose, and
wherein said increase in trabecular bone area is at least about 29 percent.

12. The method of claim 11, wherein said quercetin or at least one derivative thereof is selected from the group consisting of quercetin, avicularoside, guiajaverin, hyperoside, isohyperoside, isoquercitrin, multinoside A, multinoside A acetate, quercitrin, quercetin-3-O-(2″-O-β-D-glucopyranosyl)-α-L-rharnmopyranoside, quercetin-3-O-(6″-O-galloyl)-glucopyranoside, quercetin-3-O-(6′″-O-β-coumaroyl-β-D-glucopyranosyl-(1-2)-α-L-rhamnopyranoside), quercetin-3-O-D-glucopyranosyl-(1-6)-β-D-glucopyranosyl-(1-4)-α-L-rhamnopyranoside, quercetin-3-O-[2″-O-6′″-O-β-(7″″-O-β-D-glucopyranosyl)coumaroyl-β-D-glucopyranosyl]-α-L-rhamnopyranoside, quercetin-3-O-[6′″-β-coumaroyl-β-D-glucopyranosyl-β-(1-4)-rhamnopyranoside], quercetin-3-O-[α-L-rhamnopyranosyl (1-2)-α-L-rhamnopyranosyl-(1-6)-β-D-glucopyranoside], quercetin-3-O-[α-rhamnopyranosyl (1-4)α-L-rhamnopyranosyl (1-6)β-D-galactopyranoside], quercetin-3-O-[α-rhamnopyranosyl-(1-2)]-[β-glucopyranosyl-(1-6)]-β-D-galactopyranoside, quercetin-3-O-[α-rhamnopyranosyl-(1-4)-α-rhamnopyranosyl-(1-6)-β-galactopyranoside], quercetin-3-O-α-L-rhamnopyranosyl-(1-2)-β-D-galactopyranoside, quercetin-3-O-β-D-diglucopyranoside, quercetin-3-O-β-D-galactoside-2″-gallate, quercetin-3-O-β-D-glucopyranoside-(1-6)-β-D-galactopyranoside, quercetin-3-O-β-D-glucopyranosyl-(1-3)-α-L-rhamnopyranosyl-(1-6)-β-D-galactopyranoside, quercetin-3-O-β-D-glucuronide, quercetin-3-O-β-D-xylopyranoside, quercetin-3-O-diglucospyranoside, quercetin-3-O-gentiobioside, quercetin-3-O-glucopyranosylgalactopyranoside, quercetin-3-O-neohesperidoside, quercetin-3-gentiotrioside, quercetin-3-methyl ether, quercetin-3-rhamnogentiobioside, quercetin-3-rhamnoglucoside and quercetin-3-sulfate.

13. The method of claim 11, wherein said at least one quercetin derivative is selected from the group consisting of isorhamnetin, quercimeritrin, rhamnetin, quercetin-5-O-β-D-glucopyranoside, quercetin-7-O-β-D-glucuronopyranoside and spireaoside.

14. The method of claim 11, wherein said at least one quercetin derivative is selected from the group consisting of rhamnazin, quercetin-3′,4′-di-methyl ether, quercetin-3,3′-dimethyl ether, quercetin-3,7-dimethyl ether, quercetin-3-O-[2″-O-(6′″-O-β-coumaroyl)-β-D-glucopyranosyl]-α-L-rhamnopyranosyl-7-O-β-D-glucopyranoside, quercetin-3-O-[2″-O-6′″-O-β-(7″″-O-β-D-glucopyranosyl)coumaroyl-β-D-glucopranosyl]-α-L-rhamnopyranoside-7-O-β-D-glucopyranoside, quercetin-3-O-rutinoside-7-O-β-D-glucopyranoside, quercetin-3-O-α-L-arabinopyranosyl-7-O-β-D-glucopyranoside, quercetin-7-O-β-D-glucopyranoside-3-O-sophoroside, quercetin-3-O-galactopyranosyl-7-O-diglucopyranoside, quercetin-3-O-glucopyranosyl-7-diglucopyranoside, quercetin-3,7-diglucopyranoside, quercetin-3-gentiobiosyl-7-glucopyranoside and quercetin-3,4′-di-O-β-D-glucopyranoside.

15. The method of claim 11, wherein said at least one quercetin derivative is selected from the group consisting of quercetin-3,4′,7-trimethyl ether and quercetin-3,3′,4′,7-tetramethyl ether.
